(19) United States
(12) Patent Application Publication
(10) Pub. No.: US 2025/0241839 A1
(43) Pub. Date: Jul. 31, 2025

(54) IMPROVE THE WATER RESISTANCE OF COSMETIC COMPOSITIONS COMPRISING PIGMENTS

(71) Applicant: DSM IP Assets B.V., Maastricht (NL)

(72) Inventors: Christine MENDROK-EDINGER, Kaiseraugst (CH); Karina HECKER, Kaiseraugst (CH); Désirée Sarah HAAG, Kaiseraugst (CH)

(21) Appl. No.: 18/853,234

(22) PCT Filed: Apr. 3, 2023
(86) PCT No.: PCT/EP2023/058642

§ 371 Date: Oct. 1, 2024

(30) Foreign Application Priority Data
Apr. 4, 2022 (EP) 22166555.7

(51) Int. Cl. A61K 8/73; A61K 8/31; A61Q 1/02; A61Q 1/06; A61Q 1/08; A61Q 1/10

(52) U.S. Cl. CPC A61K 8/73; A61K 8/31; A61Q 1/02; A61Q 1/06; A61Q 1/08; A61Q 1/10; A61K 2800/43

(57) ABSTRACT

The present invention relates to the increase of water resistance of cosmetic compositions which comprise pigments. This is achieved by the combined use of a mixture of branched and linear saturated C15-C19 alkanes; and an ester of a fatty acid and dextrin.

### TECHNICAL FIELD

The present invention relates to the field of cosmetics comprising pigments, particularly the field of decorative cosmetics.

### BACKGROUND OF THE INVENTION

Cosmetic products comprise typically pigments which results in a coloured appearance when the product is applied to skin or hair. These products typically are decorative cosmetics. Decorative cosmetics are typically used to give the skin and/or hair a better visual appearance and are applied in thin layers to skin or hair, particularly in the form of a lip gloss, a lip ink, a lip stick, a makeup, a foundation, a mascara, an eye liner, an eye shadow, a liquid blush, an eyebrow liner, a concealer, a highlighter or contouring shade.

The pigments lead particularly to the visual appearance of the skin and/or hair. Due to the fact that pigments are imbedded as small particles in the product, the pigments tend to be removed rather easily from the applied cosmetic product particularly when said product is exposed to water and/or humidity and shows therefore a reduced water resistance. This is very disadvantageous as the product needs to be re-applied regularly to maintain a constant visual appearance over extended time.

### SUMMARY OF THE INVENTION

Therefore, the problem to be solved by the present invention is to improve the water resistance of cosmetic products which comprise pigments.

Surprisingly, it has been found that the cosmetic products according to claim 1 improve this water resistance significantly. Particularly, it has been found that the improvement of water resistance is improved by adding a combination of a specific mixture of branched and linear saturated C15-C19 alkanes and an ester of a fatty acid and dextrin to a cosmetic composition comprising a pigment.

Furthermore, a beneficial synergistic effect between C15-C19 alkane mixture and ester of a fatty acid and dextrin on the increase of water resistance has been found when added to a cosmetic composition which comprises at least one pigment.

Further aspects of the invention are subject of further independent claims. Particularly preferred embodiments are subject of dependent claims.

### DETAILED DESCRIPTION OF THE INVENTION

In a first aspect the present invention relates to a cosmetic composition comprising
a mixture of branched and linear saturated C15-C19 alkanes; and
an ester of a fatty acid and dextrin; and
a pigment
wherein the amount of branched saturated C15-C19 alkane in said mixture of branched and linear saturated C15-C19 alkanes is more than 80% by weight, preferably more that 90% by weight, most preferred more than 92% by weight.

In the present document, a “C_{x-y}-alkyl” group is an alkyl group comprising x to y carbon atoms, i.e., for example, a C₁₋₃-alkyl group is an alkyl group comprising 1 to 3 carbon atoms. The alkyl group can be linear or branched. For example —CH(CH₃)—CH₂—CH₃ is considered as a C₄-alkyl group.

In case identical labels for symbols or groups are present in several formulae, in the present document, the definition of said group or symbol made in the context of one specific formula applies also to other formulae which comprises the same said label.

The term “UV filter” in the present document stands for a substance that absorbs ultraviolet light (=UV light), i.e. electromagnetic radiation of the wavelength between 280 and 400 nm. UV(A) filters are UV filters that absorb UV(A) light, i.e. electromagnetic radiation of the wavelength between 315 and 400 nm. UV(B) filters are UV filters that absorb UV(B) light, i.e. electromagnetic radiation of the wavelength between 280 and 315 nm.

A liquid organic UV filter is liquid at ambient temperature (i.e. 25° C.).

A solid organic UV filter is solid at ambient temperature (i.e. 25° C.).

A “mixture of branched and linear saturated C15-C19 alkanes” in the present document means that said mixture comprises different alkanes each of them only having 15, 16, 17, 18 or 19 carbon atoms but does not comprise any alkanes having less carbons. Therefore, such a mixture does not contain for example dodecane or isododecane. Said mixture comprises both branched and linear C15-C19 alkanes.

The term “pigment” in the present document is a colored material that is completely or nearly insoluble (i.e. <100 mg/100 ml) in water at room temperature. This is to be seen in contrast to the term “dye” which is a colored material that is highly soluble in water. Pigments are small particles. Pigments can be inorganic or organic pigments.

### Mixture of Branched and Linear Saturated C15-C19 Alkanes

The cosmetic composition comprises a mixture of branched and linear saturated C15-C19 alkanes.

Particular suitable mixtures of C15-C19 alkanes are particularly the ones disclosed in WO 2016/185046, WO 2017/046177, WO 2018/109353 A1 and WO 2018/109354 A1 and WO 2018/172228 A1.

Preferably, the mixture of branched and linear saturated C15-C19 alkanes has a content of carbon of biological origin being greater or equal to 90% with respect of the total weight of the mixture of branched and linear saturated C15-C19 alkanes. The biological origin of chemicals is very advantageous as such material has a high degree of sustainability. High sustainable products or compositions are highly demanded in the market.

The determination of the content of biomaterial or content of biocarbon is given pursuant to standards ASTM D 6866-12, method B (ASTM D 6866-06) and ASTM D 7026 (ASTM D 7 026-04). Standard ASTM D 6866 concerns “Determining the Biobased Content of Natural Range Materials Using Radiocarbon and Isotope Ratio Mass Spectrometry Analysis”, while standard ASTM D 7 026 concerns “Sampling and Reporting of Results for Determination of Biobased Content of Materials via Carbon Isotope Analysis”. The second standard mentions the first in its first paragraph. The first standard describes a test of measurement of the ratio ¹⁴C/¹²C of a sample and compares it with the ratio ¹⁴C/¹²C of a sample renewable reference of origin 100%, to give a relative percentage of C of origin renewable in the sample. The standard is based on the same concepts that the dating with ¹⁴C.

It is further preferred that the composition has no or a very small amount (less than 100 ppm, particularly less than 30 ppm) of aromatic hydrocarbons with respect to the total weight of the mixture of branched and linear saturated C15-C19 alkanes.

The mixture of branched and linear saturated C15-C19 alkanes is particularly produced by catalytic hydrogenation of hydrocarbon biomass feedstock, such as described in detail in WO 2016/185046, particular the one disclosed as example 3 of WO 2016/185046.

It is preferred that the amount of linear saturated C15-C19 alkanes in said mixture of branched and linear saturated C15-C19 alkanes is less than 10% by weight, preferably less than 8% by weight, most preferred more than 5% by weight.

It is further preferred that the amount of C15 is less than 3%, particularly less than 1%, preferably less than 0.05%, by weight in respect to the weight of said mixture of branched and linear saturated C15-C19 alkanes.

It is preferred that the mixture of branched and linear saturated C15-C19 alkanes is a mixture of branched and linear saturated C16-C19 alkanes.

It is further preferred that amount of branched saturated C16-C18 alkane is more than 90% by weight, preferably more than 95% by weight in respect to the weight of said mixture of branched and linear saturated C15-C19 alkanes.

It is further preferred that the amount of C15 alkanes is less than 5%, particularly less than 2%, by weight in respect to the weight of said mixture of branched and linear saturated C15-C19 alkanes.

It is further preferred that amount of branched saturated C17-C18 alkane is more than 85% by weight, preferably more than 92% by weight in respect to the weight of said mixture of branched and linear saturated C15-C19 alkanes.

It is further preferred that the amount of C17 alkanes is more between 15 and 20% by weight in respect to the weight of said mixture of branched and linear saturated C15-C19 alkanes.

It is further preferred that amount of branched saturated C18 alkane is more than 50% by weight, preferably more than 60% by weight, even more preferably more than 70% by weight, relative to the weight of said mixture of branched and linear saturated C15-C19 alkanes.

It is further preferred that the amount of C18 alkanes is particularly between 70 and 75% by weight in respect to the weight of said mixture of branched and linear saturated C15-C19 alkanes.

In other words, the mixture of branched and linear saturated C15-C19 alkanes consist preferably mainly of C18 alkane(s), most preferably mainly of branched C18 alkane(s).

As the composition comprises a mixture of branched and linear saturated C15-C19 alkanes, said composition does not comprise any lower alkanes, i.e. it does particularly not comprise any C12 alkanes and particularly does not comprise any C12 or C13 or C14 alkanes.

It is further preferred that the mixture of C15-C19 alkanes has at 20° C., a viscosity of 3-15 mPa·s, particularly between 6 and 12 mPa·s.

It is further preferred that the mixture of C15-C19 alkanes has at 20° C. a refractive index of between 1.40 and 1.48, particularly of between 1.42 and 1.45, most preferably between 1.43 and 1.44.

It is further preferred that the mixture of C15-C19 alkanes is the mixtures of C15-C19 alkanes as commercialized as EMOGREEN™ L19 by SEPPIC.

The amount of the mixture of branched and linear saturated C15-C19 alkanes is in the range of between 0.5 and 12.0% by weight, particularly between 1.0 and 10.0% by weight, more preferably between 1.5 and 8.0% by weight, preferably between 2.0 and 6.0% by weight, based on the weight of the hair care.

### Ester of a Fatty Acid and Dextrin

The composition further comprises an ester of a fatty acid and dextrin.

Dextrin is an oligomer of D-glucose. Its structure can be represented simplified by the following structure

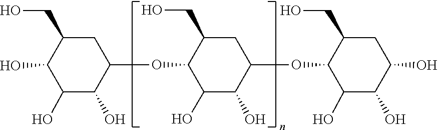

Dextrins have different average degrees of glycopolymerization which leads to different molecular weights.

In the present invention, the dextrin of said ester of a fatty acid and dextrin has preferably an average degree of glycopolymerization of between 3 and 20, particularly between 8 and 16.

It is preferred that the fatty acid of said ester of a fatty acid and dextrin is a C14-C18 fatty acid, particularly a linear C14-C18 fatty acid, most preferably palmitic acid.

As particular suitable ester of a fatty acid and dextrin is a dextrin palmitate as commercialized as Rheopearl® KL2 by Chiba Flour Milling.

Dextrin has several hydroxyl groups which can be esterified.

It is preferred that said ester of a fatty acid and dextrin has an average number of esterified hydroxyl groups of more than 2.5, preferably between 2.5 and 3.5, more preferably between 2.5 and 3.4, most preferably between 2.5 and 3.2, per glucose unit.

In one embodiment said ester of a fatty acid and dextrin has an average number of esterified hydroxyl groups of more than 2.5, preferably between 2.7 and 3.5, more preferably between 28 and 3.4, most preferably between 2.8 and 3.2, per glucose unit.

In another embodiment said ester of a fatty acid and dextrin has an average number of esterified hydroxyl groups of more than 3, preferably between 3.05 and 3.5, more preferably between 3.1 and 3.4, most preferably between 3.1 and 3.2, per glucose unit.

In other words, preferably essentially all of the hydroxyl groups of the dextrin are esterified.

It is further preferred that said ester of a fatty acid and dextrin has a molecular weight Mₙ of between 8′000 and 16′000 Da, preferably between 9′000 and 13′000 Da, more preferably between 10′000 and 11′500 Da.

The molecular weight Mn is determined in Dalton (Da) particularly by SEC/GPC using polystyrene as standard.

Both fatty acid and dextrin have biological origin. The biological origin of chemicals is very advantageous as such material or products thereof have a high degree of sustainability. High sustainable products or compositions are highly demanded in the market.

The amount of the ester of a fatty acid and dextrin is preferably in the range of between 0.1 and 15% by weight, particularly between 0.3 and 8.0% by weight, particularly between 0.6 and 7.0% by weight, more preferably between 1.0 and 6.0% by weight, particularly between 1.5 and 5.0% by weight, based on the weight of the cosmetic composition.

In the said composition, the ratio of the weight of said ester of a fatty acid and dextrin to the weight of said mixture of branched and linear saturated C15-C19 alkanes is preferably less than 1, preferably in the range of 0.5 to 0.8, most preferred in the range of 0.55-0.70.

In other words, the composition comprises preferably more, by weight, of the C15-C19 alkanes than of the ester of a fatty acid and dextrin.

### Pigment

The term “pigment” in the present document is a colored material that is completely or nearly insoluble in water or an organic solvent.

Pigments can be defined by the Colour Index number (CI). The Colour Index International is a reference database jointly maintained by the Society of Dyers and Colourists and the American Association of Textile Chemists and Colorists.

Pigments may be inorganic or organic pigments.

Inorganic Color Pigments can be for example chalk, ochre, umber, green earth, burned siena or graphite. Furthermore, inorganic color pigments, black pigments, such as, e.g., Iron oxide black, colored pigments, such as Ultramarine or iron oxide red and fluorescent or phosphorescent pigments. Particularly suitable are colored metal oxides, hydroxides and oxide hydrates, mixed-phase pigments, sulfur-containing silicates, silicates, metal sulfides, complex metal cyanides, metal sulfates, chromates and/or molybdates. Especially preferred color pigments are black iron oxide (CI 77499), yellow iron oxide (CI 77492), red and brown iron oxide (CI 77491), manganese violet (CI 77742), ultramarine (sodium aluminum sulfosilicates, CI 77007, Pigment Blue 29), chromium oxide hydrate (CI 77289), iron blue (ferric ferrocyanides, CI 77510) and/or carmine (Cochineal).

Likewise particularly preferred color pigments are colored pearlescent pigments. These are usually based on mica and mica base and may be coated with one or more metal oxides. Mica belongs to the layer silicates. The most important representatives of these silicates, muscovite, phlogopite, paragonite, biotite, lepidolith and margarit. For the production of the pearlescent pigments mica, preferably phlogopite or muscovite, is preferably coated with a metal oxide.

As an alternative to natural mica also synthetic coated mica can be used as pearlescent pigment. Particularly preferred pearlescent pigments are based on natural or synthetic mica (mica) which are coated with one or more of the aforementioned metal oxides. The color of the respective pigments may be achieved by variation of the layer thickness of the metal oxide(s).

Also preferred pigments are pigments which are based on synthetically produced mica which are coated with metal oxides, in particular based on synthetic fluoro phlogopite (INCI: Synthetic Fluoro Phlogopite). The synthetic fluoro phlogopite are, for example, coated with tin oxide, iron oxide(s) and/or titanium dioxide. The metal oxide layers, may comprise also further pigments, such as iron(III)-hexacyanidoferrat(II/III) or carmine red. Such mica pigments are, for example, commercially available under the name of the SYNCRYSTAL from Eckart.

The inorganic pigment is selected from the group of the colored metal oxides, metal hydroxides, metal oxide hydrates, silicates, metal sulfides, complex metal cyanides, metal sulfates, bronze pigments and/or colored pigments based on mica with are coated by at least one metal oxide and/or a metal oxychloride.

In a further preferred embodiment, the coloring pigments based on mica or which are coated with one or more metal oxides from the group of titanium dioxide (CI 77891), black iron oxide (CI 77499), yellow iron oxide (CI 77492), red and/or brown iron oxide (CI 77491), manganese violet (CI 77742, CI 77499), ultramarine (sodium aluminum sulfo silicates, CI 77007, Pigment Blue 29), chromium oxide hydrate (CI 77289), chromium oxide (CI 77288) and/or iron blue (ferric ferrocyanides, CI 77510).

Further suitable inorganic pigments are based on metal oxide-coated platelet-shaped borosilicates. These are, for example, tin oxide, iron oxide(s), silicon dioxide and/or titanium dioxide coated. Such borosilicate-based pigments are commercially available from ECKART, under the name MIRAGE or Reflecks from BASF SE.

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, Flamenco®, Cellini®, Cloisonneé®, Duocrome®, Gemtone®, Timica® (R)(R)(R), MultiReflections, Chione from the company BASF SE and Sunshine® from the company Sunstar.

The inorganic pigments particularly have a Colour Index number between CI 77000 and CI 77999.

In one embodiment, it is preferred that the pigment is an inorganic pigment.

Organic pigments are insoluble in water and organic solvents particularly selected from the group of nitroso, nitro, azo, xanthene, anthraquinone, isoindolinone, isoindoline, quinacridone, perinone, perylene, diketopyrrolopyorrol, thioindido, dioxazine, and/or triarylmethane compounds.

Particularly suitable as organic pigments are carmine, phthalocyanine, Sorgho, blue pigments having the Color Index numbers CI 69800, CI 69825, CI 73000, CI 74100, CI 74160; yellow pigments having the Color Index numbers CI 11680, CI 11710, CI 20040, CI 21100, CI 21108, CI 47000; green pigments having the Color Index numbers CI 61565, CI 61570, CI 74260; orange pigments having the Color Index numbers CI 11725, CI 45370, CI 71105; red pigments having the Color Index numbers CI 12085, CI 12120, CI 12370, CI 12420, CI 12490, CI 15525, CI 15800, CI 15850, CI 15865, CI 15880, CI 17200, CI 26100, CI 58000, CI 73360, CI 73915 and/or CI 75470.

In a preferred embodiment, the pigment is selected from the group consisting of CI 15850, CI 17200, CI 45370, CI 73360, CI 75470, CI 77007, CI 77013, CI 77019, CI 77120, CI 77163, CI 77266, CI 77288, CI 77289, CI 77480, CI 77491, CI 77492, CI 77499, CI 77510, CI 77713, CI 77742, CI 77820, CI 77891, CI 77947 and char coal powder.

Particularly preferred are pigments selected from the group consisting of CI 15850, CI 77491, CI 77492, CI 77499, CI 77510 and CI 77891 and char coal powder.

Particularly suitable are the pigments commercially available as Colorona® SynCranberry from Merck KGaA, Colorona® Dark Blue from Merck KGaA, Charcopowder from Cosmos, C91-4241 Intenza® Purple Reign from Sun Chemical Corp., Ronastar® Red Allure from Merck KGaA, C47-5001 SunPuro® Titanium Dioxide from Sun Chemical Corp., C33-9001 SunPuro® Yellow Iron Oxide from Sun Chemical Corp., C33-88001 SunPuro® Red Iron Oxide from Sun Chemical Corp., Unipure Black LC 989 ADT-C from Sensient Cosmetic Technologies.

Pigments particularly preferred for lip inks compositions are:

| INCI | Description |
|---|---|
| CI 15850 | Red 7 |
| CI 15850 | Red 7 lake |
| CI 17200 | D&C Red 33 |
| CI 17200 | Red 33 lake |
| CI 45370 | Orange 5 |
| CI 73360 | Red 30 |
| CI 75470 | Carmine |
| CI 77163 | Bismuth oxychloride |
| CI 77400 | Bronze Powder |
| CI 77491 | Yellow red Iron oxide |
| CI 77492 | Black Iron oxide |
| CI 77499 | Iron oxide |
| CI 77510 | Ferric ammonium ferrocyanide |
| CI 77742 | Manganese violet |
| CI 77861 | Tin oxide |
| CI 77891 | Titanium dioxide |

Pigments particularly preferred for lip gloss compositions are:

| INCI | Description |
|---|---|
| CI 12490 | D&C Red 5 |
| CI 12700 | Yellow 16 |
| CI 15525 | red/pigment red 68 |
| CI 15580 | FD&C Red No. 6 |
| CI 40800 | Beta Carotene |
| CI 69800 | Pigment Blue 60 |
| CI 75130 | Betacarotene |
| CI 77002 | Aluminium Hydroxide Sulphate |
| CI 77231 | Calciumsulfat |
| CI 77346 | Cobalt Blue Oxide |

Pigments particularly preferred for foundation compositions are:

| INCI | Description |
|---|---|
| Charcoal powder | Charcoal powder |
| CI 15850 | Red 7 |
| CI 15850 | Red 7 lake |
| CI 15850 | Red 6 |
| CI 17200 | D&C Red 33 |
| CI 17200 | Red 3 |
| CI 17200 | Red 33 lake |
| CI 73360 | Red 30 lake |
| CI 77007 | Ultramarine |
| CI 77019 | Mica |
| CI 77120 | Barium sulfate |
| CI 77163 | Bismuth oxychloride |
| CI 77288 | Chromium oxide greens |
| CI 77289 | Chromium hydroxide green |
| CI 77480 | Gold |
| CI 77491 | Red Iron oxide |
| CI 77492 | Yellow Iron oxide |
| CI 77499 | Black Iron oxide |
| CI 77510 | Ferric ammonium ferrocyanide |
| CI 77510 | Ferric ferrocyanide |
| CI 77713 | Magnesium carbonate |
| CI 77742 | Manganese violet |
| CI 77820 | Silver |
| CI 77891 | Titanium dioxide |
| CI 77947 | Zinc oxide |

Pigments particularly preferred for lip stick compositions are:

| INCI | Description |
|---|---|
| CI 12085 | D&c Red 36 |
| CI 15880 | red 34 |
| CI 15880 | red 34 lake |
| CI 40800 | Beta Carotene |
| CI 61565 | FD&C Green No. 6 |
| CI 69800 | Pigment Blue 60 |
| CI 73360 | red 30 lake |
| CI 73385 | Vat Violet 2 |
| CI 75130 | Betacarotene |
| CI 77002 | Aluminium Hydroxide Sulphate |
| CI 77004 | Kaolin |
| CI 77005 | Manganese alumina pink corundum |
| CI 77120 | Barium sulfate |
| CI 77231 | Calciumsulfat |

Pigments particularly preferred for foundation compositions are:

| INCI | Description |
|---|---|
| Charcoal powder | Charcoal powder |
| CI 15850 | Red 7 |
| CI 15850 | Red 7 lake |
| CI 15850 | Red 6 |
| CI 17200 | D&C Red 33 |
| CI 17200 | Red 3 |
| CI 17200 | Red 33 lake |
| CI 73360 | Red 30 lake |
| CI 77007 | Ultramarine |
| CI 77019 | Mica |
| CI 77120 | Barium sulfate |
| CI 77163 | Bismuth oxychloride |
| CI 77288 | Chromium oxide greens |
| CI 77289 | Chromium hydroxide green |
| CI 77480 | Gold |
| CI 77491 | Red Iron oxide |
| CI 77492 | Yellow Iron oxide |
| CI 77499 | Black Iron oxide |
| CI 77510 | Ferric ammonium ferrocyanide |
| CI 77510 | Ferric ferrocyanide |
| CI 77713 | Magnesium carbonate |
| CI 77742 | Manganese violet |
| CI 77820 | Silver |
| CI 77891 | Titanium dioxide |
| CI 77947 | Zinc oxide |

Pigments particularly preferred for mascara compositions are:

| INCI | Description |
|---|---|
| CI 77891 | Titanium dioxide |
| CI 77499 | Iron oxide |
| CI 77266 nano | Black 2 nano |
| CI 77007 | Ultramarine |
| CI 77492 | Iron oxide |
| CI 77491 | Iron oxide |
| CI 75470 | Carmine |
| CI 77288 | Chromium oxide greens |
| CI 77742 | Manganese violet |
| CI 77510 | Ferric ferrocyanide |
| CI 77289 | Chromium hydroxide green |

Pigments particularly preferred for eyeliner compositions are:

| INCI | Description |
|---|---|
| Charcoal powder | Charcoal powder |
| CI 15850 | Red 6 |
| CI 15850 | Red 7 lake |
| CI 75470 | Red 4/Carmine |
| CI 77000 | Aluminium powder |
| CI 77007 | Ultramarine |
| CI 77013 | Sodium aluminosilicate violet |
| CI 77163 | Bismuth oxychloride |
| CI 77266 | Carbon black 7 |
| CI 77266 nano | Black 2 nano |
| CI 77288 | Chromium oxide green |
| CI 77289 | Chromium hydroxide green |
| CI 77491 | Iron oxide red |
| CI 77491 | Iron oxide yellow |
| CI 77499 | Iron oxide black |
| CI 77510 | Ferric ammonium ferrocyanide |
| CI 77510 | Ferric ferrocyanide |
| CI 77742 | Manganese violet |

Pigments particularly preferred for eye shadow compositions are:

| INCI | Description |
|---|---|
| CI 15850 | Red 7 Lake |
| CI 15880 | D&C red 34 |
| CI 17200 | D&C Red 33 |
| CI 75470 | Red 4 |
| CI 75470 | Carmine |
| CI 77000 | Aluminium Powder |
| CI 77007 | Ultramarine |
| CI 77120 | Barium Sulfate |
| CI 77163 | Bismuth oxychloride |
| CI 77266 | Carbon black |
| CI 77266[Nano] | Black No. 2 D&C |
| CI 77288 | Chromium Oxide Greens |
| CI 77289 | Chromium hydroxide green |
| CI 77400 | Bronze Powder |
| CI 77400 | Copper Powder |
| CI 77491 | Red Iron oxide |
| CI 77492 | Yellow Iron oxide |
| CI 77499 | Black Iron oxide |
| CI 77510 | Ferric ammonium ferrocyanide |
| CI 77510 | Ferric ferrocyanide |
| CI 77742 | Manganese violet |
| CI 77820 | Silver |
| CI 77861 | Tin oxide |
| CI 77891 | Titanium dioxide |
| CI 77947 | Zinc Oxide |

Pigments particularly preferred for eyebrow liner compositions are:

| INCI | Description |
|---|---|
| CI 11680 | yellow 1 |
| CI 12490 | D&C Red 5 |
| CI 40800 | Beta Carotene |
| CI 77002 | Aluminium Hydroxide Sulphate |
| CI 77004 | Kaolin |
| CI 77163 | Bismuth oxychloride |
| CI 77268:1 | Black 11 |

Pigments particularly preferred for liquid blush compositions are:

| INCI | Description |
|---|---|
| CI 14815 | C-Rot 49 |
| CI 15850 | red 7 lake |
| CI 15850 | red 6 |
| CI 69825 | Vat blue 6 |
| CI 73360 | red 30 lake |
| CI 75300 | Curcumin |
| CI 77002 | Aluminium Hydroxide Sulphate |
| CI 77004 | Kaolin |
| CI 77120 | barium sulfate |
| CI 77163 | bismuth oxychloride |
| CI 77346 | Cobalt Blue Oxide |
| CI 77491 | Iron oxide |
| CI 77492 | Iron oxide |
| CI 77891 | Titanium dioxide |

Pigments particularly preferred for concealer compositions are:

| INCI | Description |
|---|---|
| CI 11680 | Yellow 1 |
| CI 12490 | Red 5 |
| CI 61565 | FD&C Green No. 6 |
| CI 77000 | Aluminium Powder |
| CI 77002 | Aluminium Hydroxide Sulphate |
| CI 77007 | Ultramarines |
| CI 77120 | Barium sulfate |
| CI 77163 | Bismuth oxychloride |
| CI 77288 | Chromium Oxide Greens |
| CI 77289 | Chromium hydroxide green |
| CI 77491 | Red Iron oxide |
| CI 77492 | Yellow Iron oxide |
| CI 77499 | Black Iron oxide |
| CI 77510 | Ferric ammonium ferrocyanide |
| CI 77510 | Ferric ferrocyanide |
| CI 77742 | Manganese violet |
| CI 77891 | Titanium dioxide |

Pigments particularly preferred for contour/highlighter compositions are:

| INCI | Description |
|---|---|
| CI 45396 | Orange 16 |
| CI 51319 | Pigment Violet 23 |
| CI 59040 | Green 8 |
| CI 61565 | FD&C Green No. 6 |
| CI 69825 | Vat blue 6 |
| CI 73360 | Red 30 lake |
| CI 75300 | Curcumin |
| CI 77120 | Barium sulfate |
| CI 77220 | Calcium Carbonate |
| CI 77231 | Calciumsulfat |
| CI 77267 | Black 3 |
| CI 77346 | Cobalt Blue Oxide |
| CI 77491 | Iron oxide |
| CI 77492 | Iron oxide |
| CI 77499 | Iron oxide |
| CI 77891 | Titanium dioxide |

The amount of the pigment is preferably in the range of between 0.001 and 15% by weight, particularly between 0.01 and 12% by weight, preferably between 0.05 and 10% by weight, based on the weight of the cosmetic composition.

The cosmetic composition can be used in any application where pigment based products are used in the field of cosmetics.

Particularly preferred the cosmetic composition is a is a decorative cosmetic composition, particularly a lip gloss, a lip ink, a lip stick, a makeup, a foundation, a mascara, an eye liner, an eye shadow, a liquid blush, an eyebrow liner, a concealer, a highlighter or contouring shade.

The cometic composition, particularly the decorative cosmetic composition, preferably the lip gloss, lip ink, lip stick, makeup, foundation, mascara, eye liner, eye shadow, liquid blush, eyebrow liner, concealer, highlighter or contouring shade may comprise further ingredients which are known to be typically used in said specific product types.

The cometic composition comprises particularly water and may be in the form of a suspension or dispersion in solvents or fatty substances, or alternatively in the form of an emulsion or micro emulsion (in particular of oil-in-water (O/W-) or water-in-oil (W/O-)type, silicone-in-water (Si/W-) or water-in-silicone (W/Si-)type, PIT-emulsion, multiple emulsion (e.g. oil-in-water-in oil (O/W/O-) or water-in-oil-in-water (W/O/W-)type), pickering emulsion, hydrogel, alcoholic gel, lipogel, one- or multiphase solution or vesicular dispersion.

Preferred cosmetic compositions in all embodiments of the present invention preferably comprise water and are in the form of an emulsion.

The emulsion particularly contains an oily phase and an aqueous phase such as in particular O/W, W/O, Si/W, W/Si, O/W/O, W/O/W multiple or a pickering emulsions.

The total amount of the oily phase present in such emulsions is preferably at least 10 wt.-%, such as in the range from 10 to 60 wt.-%, preferably in the range from 15 to 50 wt.-%, most preferably in the range from 15 to 40 wt.-%, based on the total weight of the cosmetic composition.

The amount of the aqueous phase present in such emulsions is preferably at least 20 wt. %, such as in the range from 40 to 90 wt.-%, preferably in the range from 50 to 85 wt.-%, most preferably in the range from 60 to 85 wt.-%, based on the total weight of the cosmetic composition.

More preferably, the cosmetic compositions are in the form of an oil-in-water (O/W) emulsion comprising an oily phase dispersed in an aqueous phase in the presence of an O/W- respectively Si/W-emulsifier. The preparation of such O/W emulsions is well known to a person skilled in the art.

The compositions in form of O/W emulsions can be provided, for example, in all the formulation forms for O/W emulsions, for example in the form of serum, milk or cream, and they are prepared according to the usual methods. In case, the cosmetic composition is a decorative product, the cosmetic product is rather a pasty dispersion.

The cosmetic composition comprises preferably a UV filter. The further UV filter may be solid or liquid. It is preferred that the further UV filter is a solid UV filter.

Suitable liquid organic UV-filter absorb light in the UV(B) and/or UV(A) range and are liquid at ambient temperature (i.e. 25° C.). Such liquid UV-filter are well known to a person in the art and encompass in particular cinnamates such as e.g. octyl methoxycinnamate (PARSOL® MCX) and isoamyl methoxycinnamate (Neo Heliopan® E 1000), salicylates such as e.g. homosalate (3,3,5 trimethyl-cyclohexyl 2-hydroxybenzoate, PARSOL® HMS) and ethylhexyl salicylate (also known as ethylhexyl salicylate, 2-ethylhexyl-2-hydroxybenzoate, PARSOL® EHS), acrylates such as e.g. octocrylene (2-ethylhexyl-2-cyano-3,3-diphenylacrylate, PARSOL® 340) and ethyl 2-cyano-3,3 diphenylacrylate, esters of benzalmalonic acid such as in particular dialkyl benzalmalonates such as e.g. di (2-ethylhexyl) 4-methoxybenzalmalonate and polysilicone 15 (PARSOL® SLX), dialkylester of naphthalates such as e.g. diethylhexyl 2,6-naphthalate (Corapan® TQ), syringylidene malonates such as e.g. diethylhexyl syringylidene malonate (Oxynex® ST liquid) as well as benzotriazolyl dodecyl p-cresol (Tinoguard® TL) as well as benzophenone-3 and drometrizole trisiloxane.

Particular advantageous liquid organic UV-filter are octyl methoxycinnamate, homosalate, ethylhexyl salicylate, octocrylene, diethylhexyl 2,6-naphthalate, diethylhexyl syringylidene malonate, benzotriazolyl dodecyl p-cresol, benzo-phenone-3, drometrizole trisiloxane as well as mixtures thereof.

In a preferred embodiment, the liquid UV filter is a liquid UV(B) filter which is selected from the group consisting of ethylhexyl methoxycinnamate, octocrylene, homosalate, ethylhexyl salicylate, benzophenone-3 and drometrizole trisiloxane.

Suitable solid organic UV-filter absorb light in the UV(B) and/or UV(A) range and are solid at ambient temperature (i.e. 25° C.). Particularly suited solid UV-filters are of the group consisting of bis-ethylhexyloxyphenol methoxyphenyl triazine, butyl methoxydibenzoyl methane, diethylamino hydroxybenzoyl hexyl benzoate, ethylhexyl triazone, diethylhexyl butamido triazone, 4-methylbenzylidene camphor and 1,4-di(benzoxazol-2′-yl)benzene, methylene bis-benzotriazolyl tetramethylbutylphenol (Bisoctrizole) and Bemotrizinol (=bis-ethylhexyloxyphenol methoxyphenyl triazine (INCI)), particularly as commercially available as Parsol® Shield from DSM Nutritional Products Ltd.

Bisoctrizole is a broad-spectrum ultraviolet radiation filter, absorbing UV(B) as well as UV(A) rays and has an excellent photostability. It has an absorption maximum at 308 nm and 349 nm. However, next to absorption of UV light, it also reflects and scatters UV light. Therefore, Bisoctrizole is a hybrid UV absorber, an organic UV filter produced in microfine organic particles (<200 nm). Where other organic UV filters need to be dissolved in either the oil or water phase, bisoctrizole dissolves poorly in both and is applied as invisible particle.

A preferred solid organic UV(A) filter is a UV(A) filter which is selected from the group consisting of bis-ethylhexyloxyphenol methoxyphenyl triazine, butyl methoxydibenzoyl methane, diethylamino hydroxybenzoyl hexyl benzoate and tris-biphenyl triazine.

A preferred solid organic UV(B) filter is a UV(B) filter which is selected from the group consisting of ethylhexyl triazone (=Uvinul® T150), diethylhexyl butamido triazone (=Uvasorb® HEB), and 4-methylbenzylidene camphor (=PARSOL® 5000).

The cosmetic composition, furthermore, comprises preferably a moisturizer or skin humectant, particularly glycerol and/or panthenol and/or propandiol, and/or Pentavitin, particularly as commercially available as D-Panthenol 75 L, or Tilamar® PDO with Nøøvista or Pentavitin® from DSM Nutritional Products Ltd.

The cosmetic composition further comprises preferably a sensory enhancer, particularly silica, preferably as commercially available as Valvance® Touch 210 from DSM Nutritional Products Ltd.

The cosmetic composition, furthermore, may contain the usual oily and fatty components which may be chosen from mineral oils and mineral waxes; oils such as triglycerides of caprinic acid and/or caprylic acid or castor oil; oils or waxes and other natural or synthetic oils, in a preferred embodiment esters of fatty acids with alcohols e.g. isopropanol, propyleneglycol, glycerin or esters of fatty alcohols with carbonic acids or fatty acids; alkylbenzoates; and/or silicone oils.

Exemplary fatty substances which can be particularly incorporated in the oil phase of the emulsion, microemulsion, oleo gel, hydrodispersion or lipodispersion are advantageously chosen from esters of saturated and/or unsaturated, linear or branched alkyl carboxylic acids with 3 to 30 carbon atoms, and saturated and/or unsaturated, linear and/or branched alcohols with 3 to 30 carbon atoms as well as esters of aromatic carboxylic acids and of saturated and/or unsaturated, linear or branched alcohols of 3-30 carbon atoms. Such esters can advantageously be selected from octylpalmitate, octylcocoate, octylisostearate, octyldodecylmyristate, cetearylisononanoate, isopropylmyristate, isopropylpalmitate, isopropylstearate, isopropyloleate, n-butylstearate, n-hexyllaurate, n-decyloleate, isooctylstearate, isononylstearate, isononylisononanoate, 2-ethyl hexylpalmitate, 2-ethylhexyllaurate, 2-hexyldecylstearate, 2-octyldodecylpalmitate, stearylheptanoate, oleyloleate, oleylerucate, erucyloleate, erucylerucate, tridecylstearate, tridecyltrimellitate, as well as synthetic, half-synthetic or natural mixtures of such esters e.g. jojoba oil.

Other fatty components, particularly suitable for hair conditioner, include polar oils such as lecithins and fatty acid triglycerides, namely triglycerol esters of saturated and/or unsaturated, straight or branched carboxylic acid with 8 to 24 carbon atoms, preferably of 12 to 18 carbon-atoms whereas the fatty acid triglycerides are preferably chosen from synthetic, half synthetic or natural oils (e.g. cocoglyceride, olive oil, sun flower oil, soybean oil, peanut oil, rape seed oil, sweet almond oil, palm oil, coconut oil, castor oil, hydrogenated castor oil, wheat oil, grape seed oil, macadamia nut oil and others); apolar oils such as linear and/or branched hydrocarbons and waxes e.g. mineral oils, vaseline (petrolatum); paraffins, squalane and squalene, polyolefins, hydrogenated polyisobutenes and isohexadecanes, favored polyolefins are polydecenes; dialkyl ethers such as dicaprylylether; linear or cyclic silicone oils such as preferably cyclomethicone (octamethylcyclotetrasiloxane; cetyldimethicone, hexamethylcyclotrisiloxane, polydimethylsiloxane, poly(methylphenylsiloxane) and mixtures thereof.

Other fatty components which can advantageously be incorporated in the cosmetic composition are isoeikosane; neopentylglycoldiheptanoate; propylene-glycoldicaprylate/dicaprate; caprylic/capric/diglycerylsuccinate; butyleneglycol caprylat/caprat; C₁₂₋₁₃-alkyllactate; di-C₁₂₋₁₃-alkyltartrate; triisostearin; dipentaerythrityl hexacaprylat/hexacaprate; propyleneglycolmonoisostearate; tricaprylin; dimethylisosorbid. Especially beneficial is the use of mixtures C₁₂₋₁₅-alkyl-benzoate and 2-ethylhexylisostearate, mixtures C₁₂₋₁₅-alkylbenzoate and isotridecylisononanoate as well as mixtures of C₁₂₋₁₅-alkylbenzoate, 2-ethylhexyl-isostearate and isotridecylisononanoate.

The oily phase of the cosmetic composition can also contain natural vegetable or animal waxes such as bees wax, china wax, bumblebee wax and other waxes of insects as well as shea butter and cocoa butter.

Suitable silicone oils are e.g. dimethylpolysiloxane, diethylpolysiloxane, diphenylpolysiloxane, cyclic siloxanes, poly(methylphenylsiloxanes) as well as amino-, fatty acid-, alcohol-, polyether-, epoxy-, fluor-, glycoside-, and/or alkyl modified silicone compounds which are liquid or solid at room temperature and mixtures thereof. The number average molecular weight of the dimethicones and poly (methylphenylsiloxanes) is preferably in the range of 100 to 150′000 g/mol. Preferred cyclic siloxanes comprise 4- to 8-membered rings which are for example commercially available as cyclomethicones.

In one advantageous embodiment, the cosmetic compositions in addition contain a phosphate ester emulsifier. Among the preferred phosphate ester emulsifier are C8-10 Alkyl Ethyl Phosphate, C9-15 Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C12-15 Pareth-2 Phosphate, C12-15 Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular phosphate ester emulsifier is potassium cetyl phosphate e.g. commercially available as Amphisol® K at DSM Nutritional Products Ltd Kaiseraugst.

The cosmetic composition further may comprise cosmetic carriers, excipients and diluents as well as additives and active ingredients commonly used in the skin care industry which are suitable for use in the cosmetic compositions are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

Such possible ingredients of the cosmetic composition are particularly enhance the performance and/or consumer acceptability such as preservatives, antioxidants, fatty substances/oils, thickeners, softeners, light-screening agents, moisturizers, fragrances, co-surfactants, fillers, sequestering agents, cationic-, nonionic-or amphoteric polymers or mixtures thereof, acidifying or basifying agents, viscosity modifiers, and natural hair nutrients such as botanicals, fruit extracts, sugar derivatives and/or amino acids or any other ingredients usually formulated into cosmetic compositions. The necessary amounts of the adjuvants and additives can, based on the desired product, easily be chosen by a person skilled in the art in this field and will be illustrated in the examples, without being limited hereto.

The cosmetic compositions preferably have a pH in the range from 3 to 10, preferably a pH in the range from 4 to 8 and most preferably a pH in the range from 4 to 7.5. The pH can easily be adjusted as desired with suitable acids such as e.g. citric acid or bases such as NaOH according to standard methods in the art.

In one embodiment, the composition is sulfate-free. The term “free” as used in the present document, for example in “sulfate-free”, is used to mean that the respective substance is only present at amounts of less than 0.5% by weight, particularly less than 0.1% by weight, more particularly below 0.05% by weight, relative to the weight of the composition. Preferably, “free” means that the respective substance is completely absent in the composition.

It has been shown that the cosmetic compositions have a significantly higher water resistance as compared to the respective compositions without a mixture of branched and linear saturated C15-C19 alkanes and ester of a fatty acid and dextrin.

It has been shown that the combined use of C15-C19 alkane mixture and ester of a fatty acid and dextrin leads to a significantly higher water resistance than when C15-C19 alkanes and ester of a fatty acid and dextrin alone. This is even more surprising as the use of the C15-C19 alkane mixture alone (i.e. without the ester of dextrin ad fatty acid) even decreases the water resistance, particularly in extended exposure to water and/or humidity. Therefore, a beneficial synergistic effect between the C15-C19 alkane mixture and the ester of a fatty acid and dextrin on the increase of water resistance of cosmetic composition comprising at least one pigment has been found.

The water resistance may be assessed by measuring the color changes after exposure of the applied cosmetic compositions to water.

One of suitable methods of color changes is the representation of color by the so-called Lab color space. Currently the mostly used Lab method is the CIE-L*a*b* as proposed by CIE in 1976. In this representation the parameters “L*”, is lightness, and the parameters “a*”, and “b*”, are color-opponent dimensions. The parameter L*, has a value between 0 and 100, parameters a* and b*, have a value between −100 and 100.

Changes in color are, hence, defined by a change of L* (=ΔL*), a* (=Δa*), and b* (=Δb*) values.

Despite the background theory is rather complex, a decrease of value of L* leads to a sensation of a darker color. An increase of the value a* leads to a sensation of enhancing of the red color component. An increase of the value b* leads to a sensation of enhancing of the yellow color component.

A color change is usually not only a change of an individual parameter L*, a* or b*, but a change of more than one of these parameters simultaneously.

Hence, there has been the color difference defined which is expressed by ΔE*, according to the following formula

$$Δ {E}^{*} = \sqrt{Δ {L}^{* 2} + Δ {a}^{* 2} + Δ {b}^{* 2}}$$

It has been shown that particularly for black colored cosmetic compositions, such as for mascara compositions, the measurement of opacity the other hand gives a good indication of water resistance.

Hence, in a further aspect, the invention relates to the use of a mixture of branched and linear saturated C15-C19 alkanes, in combination with an ester of a fatty acid and dextrin, as discussed already above in great detail, for increasing the water resistance of a cosmetic composition comprising a pigment.

The cosmetic composition is preferably a decorative cosmetic composition, particularly a lip gloss, a lip ink, a lip stick, a makeup, a foundation, a mascara, an eye liner, an eye shadow, a liquid blush, an eyebrow liner, a concealer or a highlighter or contouring shade, as already discussed above.

### EXAMPLES

The following examples are provided to further illustrate the compositions and effects of the present invention. These examples are illustrative only and are not intended to limit the scope of the invention in any way.

The cosmetic composition as outlined in table 1 or table 3 have been prepared according to standard methods in the art.

TABLE 1
Lip ink composition (all ingredients in % by weight)

| Ingredient | INCI name | Ref. 1 | Ref. 2 | Ref. 3 | 1 |
|---|---|---|---|---|---|
| Water | AQUA | 53.48 | 55.48 | 53.48 | 53.48 |
| TILAMAR ® PDO with | PROPANEDIOL | 15.00 | 15.00 | 15.00 | 15.00 |
| NØØVISTA ™ | | | | | |
| Dermosoft ® Octiol | CAPRYLYL GLYCOL | 0.20 | 0.20 | 0.20 | 0.20 |
| Cosphaderm ® Pentiol | PENTYLENE GLYCOL | 3.00 | 3.00 | 3.00 | 3.00 |
| natural | | | | | |
| Sepiplus 400 | POLYACRYLATE-13; POLYISOBUTENE; | 2.00 | 2.00 | 2.00 | 2.00 |
| | POLYSORBATE 20 | | | | |
| AMPHISOL ® K | POTASSIUM CETYL PHOSPHATE | 0.50 | 0.50 | 0.50 | 0.50 |
| Dermol 99 | SONONYL ISONONANOATE | 3.00 | 3.00 | 6.00 | 3.00 |
| Dowsil FA 4003 DM | DIMETHICONE; ACRYLATES/POLYTRIMETHYL- | 5.00 | | | |
| Silicone Acrylate | SILOXYMETHACRYLATE COPOLYMER | | | | |
| Emogreen ™ L19 | C15-19 ALKANE | | 3.00 | | 3.00 |
| Dextrin palmitate¹ | DEXTRIN PALMITATE | | | 2.00 | 2.00 |
| Colorona ® SynCranberry | SYNTHETIC FLUORPHLOGOPITE; CI 77491 | 9.95 | 9.95 | 9.95 | 9.95 |
| Charcopowder | CHARCOAL POWDER | 1.35 | 1.35 | 1.35 | 1.35 |
| C91-4241 Intenza ® Purple | CI 15850; SYNTHETIC FLUORO- PHLOGOPITE; | 1.75 | 1.75 | 1.75 | 1.75 |
| Reign | CI 77891 | | | | |
| Colorona ® Dark Blue | MICA; CI 77510; CI 77891 | 0.96 | 0.96 | 0.96 | 0.96 |
| Ronastar ® Red Allure | CI 77491; SILICA; CI 77891; MICA | 0.75 | 0.75 | 0.75 | 0.75 |
| Citric Acid 25% solution | AQUA; CITRIC ACID | 3.06 | 3.06 | 3.06 | 3.06 |

¹Dextrin Palmitate: Mₙ = 11′300-11′500 Da, determined by SEC/GPC

TABLE 2
Foundation composition (all ingredients in % by weight)

| Ingredient | INCI Name | Ref. 4 | Ref. 5 | Ref. 6 | 2 |
|---|---|---|---|---|---|
| X-Tend 226 | PHENETHYL BENZOATE | 6.00 | 6.00 | 6.00 | 6.00 |
| PARSOL ® EHS | ETHYLHEXYL SALICYLATE | 3.00 | 3.00 | 3.00 | 3.00 |
| KAK TCIN | TRICYCLODECANEMETHYL ISONONANOATE | 2.00 | 2.00 | 2.00 | 2.00 |
| Finsolv ® TN | C12-15 ALKYL BENZOATE | 1.00 | 1.00 | 4.00 | 1.00 |
| Lanette ® O | CETEARYL ALCOHOL | 0.80 | 0.80 | 0.80 | 0.80 |
| PARSOL ® SLX | POLYSILICONE-15 | 1.50 | 1.50 | 1.50 | 1.50 |
| PARSOL ® Shield | BIS-ETHYLHEXYLOXYPHENOL | 2.00 | 2.00 | 2.00 | 2.00 |
| | METHOXYPHENYL TRIAZINE | | | | |
| PARSOL ® TX | TITANIUM DIOXIDE; SILICA; DIMETHICONE | 2.50 | 2.50 | 2.50 | 2.50 |
| AMPHISOL ® K | POTASSIUM CETYL PHOSPHATE | 3.00 | 3.00 | 3.00 | 3.00 |
| TEGIN ® M Pellets MB | GLYCERYL STEARATE | 2.00 | 2.00 | 2.00 | 2.00 |
| C47-5001 SunPuro ® | CI 77891 | 3.58 | 3.58 | 3.58 | 3.58 |
| Titanium Dioxide | | | | | |
| C33-9001 SunPuro ® | CI 77492 | 0.50 | 0.50 | 0.50 | 0.50 |
| Yellow Iron Oxide | | | | | |
| Charcopowder | CHARCOAL POWDER | 0.06 | 0.06 | 0.06 | 0.06 |
| C33-88001 SunPuro ® Red | CI 77491 | 0.07 | 0.07 | 0.07 | 0.07 |
| Iron Oxide | | | | | |
| Emogreen ™ L19 | C15-19 ALKANE | | 3.00 | | 3.00 |
| Dextrin palmitate¹ | DEXTRIN PALMITATE | | | 2.00 | 2.00 |
| euxyl ® pe 9010 | PHENOXYETHANOL; ETHYLHEXYLGLYCERIN | 1.00 | 1.00 | 1.00 | 1.00 |
| VALVANCE ® Touch 210 | SILICA | 0.50 | 0.50 | 0.50 | 0.50 |
| DUB DIS | DIISOPROPYL SEBACATE | 1.20 | 1.20 | 1.20 | 1.20 |
| Water Dem | AQUA | 56.17 | 53.17 | 51.17 | 51.17 |
| Edeta ® BD | DISODIUM EDTA | 0.10 | 0.10 | 0.10 | 0.10 |
| Keltrol ® CG-T | XANTHAN GUM | 0.30 | 0.30 | 0.30 | 0.30 |
| STRUCTURE ® XL | HYDROXYPROPYL STARCH PHOSPHATE | 2.00 | 2.00 | 2.00 | 2.00 |
| Water Dem | AQUA | 5.00 | 5.00 | 5.00 | 5.00 |
| Sodium Hydroxide 30% | AQUA; SODIUM HYDROXIDE | 1.35 | 1.35 | 1.35 | 1.35 |
| Solution | | | | | |
| PARSOL ® HS | PHENYLBENZIMIDAZOLE SULFONIC ACID | 1.50 | 1.50 | 1.50 | 1.50 |
| PENTAVITIN ® | SACCHARIDE ISOMERATE; AQUA; CITRIC | 1.50 | 1.50 | 1.50 | 1.50 |
| | ACID; SODIUM CITRATE | | | | |
| PEPHA ®-TIGHT | ALGAE EXTRACT; PULLULAN; AQUA; | 1.00 | 1.00 | 1.00 | 1.00 |
| | PHENOXYETHANOL; SODIUM BENZOATE; | | | | |
| | POTASSIUM SORBATE | | | | |
| Sodium Hydroxide 30% | AQUA; SODIUM HYDROXIDE | 0.37 | 0.37 | 0.37 | 0.37 |
| Solution | | | | | |

¹Dextrin Palmitate: Mₙ = 11′300-11′500 Da, determined by SEC/GPC

TABLE 3
Mascara composition (all ingredients in % by weight)

| Ingredient | INCI Name | Ref. 7 | 3 |
|---|---|---|---|
| Paraffinum Perliquidum Ph. Eur. | PARAFFINUM LIQUIDUM | 5.00 | 5.00 |
| Carnauba wax T1 Flakes | COPERNICIA CERIFERA CERA | 3.50 | 3.50 |
| Kahlwax 8104 | CERA ALBA | 5.00 | 5.00 |
| Ecorol 16/98P | CETYL ALCOHOL | 5.00 | 5.00 |
| AMPHISOL ® K | POTASSIUM CETYL PHOSPHATE | 3.00 | 3.00 |
| Emogreen ™ L19 | C15-19 ALKANE | | 3.00 |
| Dextrin palmitate¹ | DEXTRIN PALMITATE | | 2.00 |
| Eastman AQ Polymer 55 S | POLYESTER-5 | 5.00 | |
| Water Dem | AQUA | 61.65 | 61.65 |
| Natrosol 250 HR PC | HYDROXYETHYLCELLULOSE | 0.50 | 0.50 |
| Glycerin 99.5% | GLYCERIN | 3.00 | 3.00 |
| SOLAGUM(TM) AX | ACACIA SENEGAL GUM; XANTHAN GUM | 0.20 | 0.20 |
| Unipure Black LC 989 ADT-C | CI 77499; ISOPROPYL TITANIUM TRIISOSTEARATE; | 6.00 | 6.00 |
| | BIS-PEG-15 DIMETHICONE/IPDI COPOLYMER; PEG-2 | | |
| | SOYAMINE | | |
| D-Panthenol 75 L | PANTHENOL; AQUA | 0.50 | 0.50 |
| Phytantriol | PHYTANTRIOL | 0.50 | 0.50 |
| Euxyl ® PE 9010 | PHENOXYETHANOL; ETHYLHEXYLGLYCERIN | 1.00 | 1.00 |
| Sodium Hydroxide Pellets Emprove | SODIUM HYDROXIDE | 0.15 | 0.15 |

¹Dextrin Palmitate: Mₙ = 11′300-11′500 Da, determined by SEC/GPC

### Water Resistance (Measurement of L* a* b*)

1.3 mg/cm² of the respective compositions as outlined in tables 1 and 2 and 3 were applied to 4 PMMA plates (Schönberg, 5 μm) and the plates were dried at 40° C. for 30 min. Afterwards the initial L* a* b* values were determined with a Konica Minolta-CM 600d color measurement device. 5 measurement points have been done per plate and the average L* a* b* values per plate and for all 4 plates have been calculated. Said values are given in tables 3 and 4 and 5 (“Original”). Then the plates were immersed into a flask filled with 4 l of water (bi-distilled) for 20 min while the water was stirred with a paddle agitator at 150 min⁻¹ at a water temperature of 30° C. (The plates were attached at the edge of the flask with a clothespin, such that the side covered with the composition was directed into the flask). Afterwards the plates were dried at 40° C. for 30 min. Again the L* a* b* values were determined, 5 measurement points per plate and the average L* a* b* values of all 4 plates have been calculated and given in tables 3 and 4 and 5 (“After 1 cycle”). The plates have then treated with water as described above for another water exposure cycle. The values of said second water exposure cycle are also given in tables 3 and 4 and 5 (“After 2 cycles”).

After each cycle the differences of the L* a*b* to the original values have been calculated as ΔL*, ΔLa*, ΔLb* from which the ΔE* have been determined

$$Δ {E}^{*} = \sqrt{Δ {L}^{* 2} + Δ {a}^{* 2} + Δ {b}^{* 2}}$$

The lower the ΔE* is, the less color change has occurred by the water exposure, and, hence, less pigments have been removed from the composition film.

TABLE 4
Measurement of L*a*b* values and ΔE* as indication of
water resistance of the respective Lip ink compositions.

| | | Ref. 1 | Ref. 2 | Ref. 3 | 1 |
|---|---|---|---|---|---|
| Original | L* | 50.73 | 50.1 | 49.59 | 50.97 |
| | a* | 15.13 | 19.08 | 21.11 | 17.13 |
| | b* | 7.44 | 4.32 | 5.40 | 7.28 |
| After 1 cycle | L* | 68.53 | 66.38 | 60.28 | 62.22 |
| | a* | 8.3 | 8.75 | 14.6 | 15.17 |
| | b* | 2.99 | 2.21 | 4.78 | 3.99 |
| | ΔL* | 17.8 | 16.28 | −10.69 | 11.25 |
| | Δa* | −6.83 | −10.34 | −6.51 | −1.97 |
| | Δb* | −4.44 | −2.11 | −0.62 | −3.29 |
| | ΔE* | 19.57 | 19.4 | 12.53 | 11.89 |
| | | | −0.9%^{‡} | −36.0%^{‡} | −39.2%^{‡} |
| After 2 cycles | L* | 68.98 | 68.59 | 62.25 | 62.34 |
| | a* | 8.27 | 7.67 | 13.12 | 13.79 |
| | b* | 2.97 | 1.25 | 4.33 | 3.87 |
| | ΔL* | 18.25 | 18.49 | 12.66 | 11.37 |
| | Δa* | −6.86 | −11.41 | −8.00 | −3.34 |
| | Δb* | −4.46 | −3.07 | −1.07 | −3.41 |
| | ΔE* | 20 | 21.94 | 15.01 | 12.33 |
| | | | 9.7%^{‡} | −25.0%^{‡} | −38.4%^{‡} |

^{‡}Changes in ΔE* values

TABLE 5
Measurement of L*a*b* values and ΔE* as indication of
water resistance of the respective foundation compositions.

| | | Ref. 4 | Ref. 5 | Ref. 6 | 2 |
|---|---|---|---|---|---|
| Original | L* | 72.29 | 72.11 | 72.22 | 71.85 |
| | a* | 2.80 | 2.62 | 2.56 | 2.99 |
| | b* | 11.00 | 10.48 | 9.95 | 11.40 |
| After 1 cycle | L* | 76.11 | 77.19 | 74.03 | 73.00 |
| | a* | 1.98 | 1.64 | 2.24 | 2.60 |
| | b* | 4.48 | 0.84 | 7.63 | 9.55 |
| | ΔL* | 3.82 | 5.08 | −1.81 | 1.15 |
| | Δa* | −0.82 | −0.98 | −0.32 | −0.39 |
| | Δb* | −6.52 | −9.64 | −2.32 | −1.85 |
| | ΔE* | 7.60 | 10.94 | 2.96 | 2.21 |
| | | | 44.0%^{‡} | −61.1%^{‡} | −70.9%^{‡} |
| After 2 cycles | L* | 76.17 | 77.21 | 75.51 | 73.27 |
| | a* | 1.96 | 1.59 | 2.07 | 2.56 |
| | b* | 3.55 | 0.71 | 6.41 | 8.73 |
| | ΔL* | 3.88 | 5.10 | 3.29 | 1.42 |
| | Δa* | −0.84 | −1.03 | −0.49 | −0.43 |
| | Δb* | −7.45 | −9.77 | −3.54 | −2.67 |
| | ΔE* | 8.44 | 11.06 | 4.86 | 3.05 |
| | | | 31.1%^{‡} | −42.4%^{‡} | −63.8%^{‡} |

^{‡}Changes in ΔE* values

TABLE 6
Measurement of L*a*b* values and ΔE* as indication of
water resistance of the respective mascara compositions.

| | | Ref. 7 | 3 |
|---|---|---|---|
| Original | L* | 36.03 | 30.67 |
| | a* | 0.83 | 0.57 |
| | b* | 0.43 | 0.76 |
| After 1 cycle | L* | 34.63 | 31.87 |
| | a* | 0.88 | 0.62 |
| | b* | 0.76 | 0.60 |
| | ΔL* | −1.40 | 1.19 |
| | Δa* | 0.05 | 0.05 |
| | Δb* | 0.33 | −0.17 |
| | ΔE* | 1.44 | 1.21 |
| | | | −16.0%^{‡} |
| After 2 cycles | L* | 42.44 | 35.81 |
| | a* | 0.93 | 0.50 |
| | b* | 0.17 | 0.24 |
| | ΔL* | 6.41 | 5.14 |
| | Δa* | 0.10 | −0.06 |
| | Δb* | −0.26 | −0.52 |
| | ΔE* | 6.42 | 5.17 |
| | | | −19.5%^{‡} |

^{‡}Changes in ΔE* values

In all cases the cosmetic composition according to the invention showed a significantly higher water resistance and the colour (due to the pigments) has less changed (ΔE) after one or several cycles of water exposure.

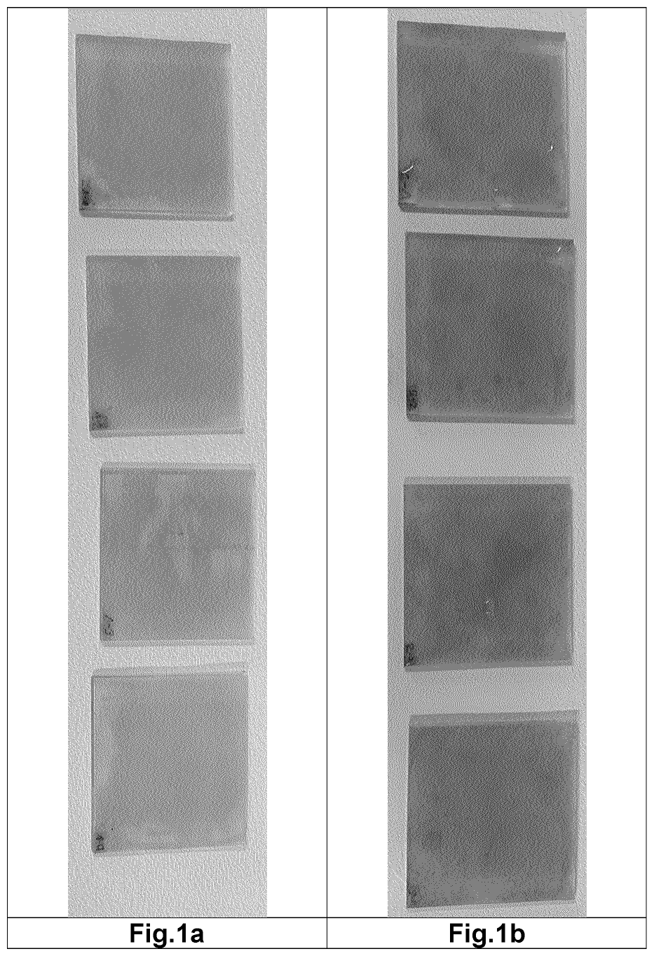

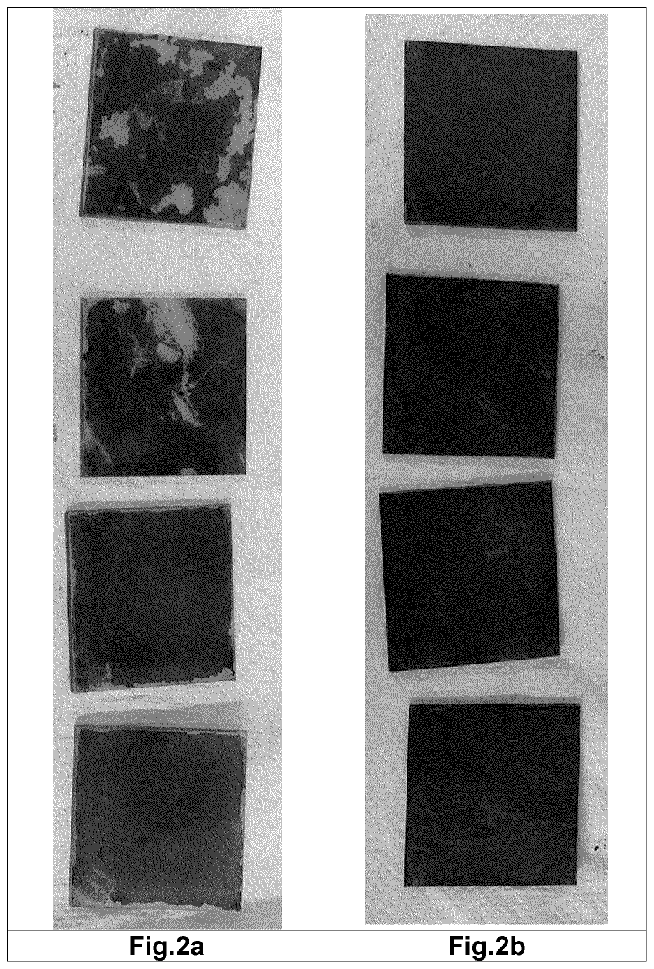

In the case of lip ink and foundation the layer remained homogenously on the plate. This is shown exemplary for example for Ref.1 in FIG. 1a and for 1 (in FIG. 1b after 2 cycles. In case of the mascara compositions this was only the case for the mascara composition according to the invention (i.e. 3) as shown in FIG. 2b) whereas the mascara composition of comparison (i.e. Ref.7) showed significant detachments as shown in FIG. 2a.

Therefore, the opacity of the films after 2 cycles of water exposure have been measured.

The opacity was measured using a Minolta 3600d Spectrophotometer Instrument setting:
Reflectance
Specular Component: SCI
Measurement Aera: MAV (25.4 mm)
UV setting (100%)

For each test formulations (i.e. Ref.7 and 3) the 4 PMMA plates have been taken after the second water exposure cycle and to measure the reflectance of the PMMA plate, the specimen was secured with the sample holder. 4 measurements on each plate have been done to cover a maximum of surface. Lightness was measured both on black (L*_{black}) and white (L*_{white}) backgrounds to obtain the contrast rate. Thus, 4 measurements have been done with a black background and 4 measurements with a white background on each PMMA plate. The opacity, shown in table 7, was calculated according to the equation based on the mean of the respective lightness values:

$$Opacity ( \% ) = contrast rate = \frac{{L}_{black}^{*}}{{L}_{white}^{*}} * 100$$

The higher the opacity is the darker the composition is and, hence, less black pigment have been removed by the water exposure.

TABLE 7
Opacity of mascara (Ref. 7 and 3) after 2 cycles of water exposure.

| | Ref. 7 | 3 |
|---|---|---|
| L*_{black} | 34.73 | 35.56 |
| L*_{white} | 44.44 | 38.32 |
| Opacity [%] | 78.15 | 92.82 |
| | | +18.77%^{‡} |

^{‡}Changes in opacity

This method as well shows that the composition of invention has a significant higher opacity, and, hence, a higher water resistance.

1. A cosmetic composition comprising
a mixture of branched and linear saturated C15-C19 alkanes; and
an ester of a fatty acid and dextrin; and
a pigment
wherein the amount of branched saturated C15-C19 alkane in said mixture of branched and linear saturated C15-C19 alkanes is more than 80% by weight, preferably more that 90% by weight, most preferred more than 92% by weight.

2. The cosmetic composition according to claim 1, wherein the amount of linear saturated C15-C19 alkanes in said mixture of branched and linear saturated C15-C19 alkanes is less than 10% by weight, preferably less than 8% by weight, most preferred more than 5% by weight.

3. The cosmetic composition according to claim 1, wherein the amount of branched saturated C18 alkane is more than 50% by weight, preferably more than 60% by weight, even more preferably more than 70% by weight, relative to the weight of said mixture of branched and linear saturated C15-C19 alkanes.

4. The cosmetic composition according to claim 1, wherein the fatty acid of said ester of a fatty acid and dextrin is a C14-C18 fatty acid, particularly a linear C14-C18 fatty acid, most preferably palmitic acid.

5. The cosmetic composition according to claim 1, wherein the dextrin of said ester of a fatty acid and dextrin has an average degree of glycopolymerization of between 3 and 20, particularly between 8 and 16.

6. The cosmetic composition according to claim 1, wherein said ester of a fatty acid and dextrin has an average number of esterified hydroxyl groups of more than 2.5, preferably between 2.5 and 3.5, more preferably between 2.5 and 3.4, most preferably between 2.5 and 3.2, per glucose unit.

7. The cosmetic composition according to claim 1, wherein said ester of a fatty acid and dextrin has a molecular weight Mₙ of between 8′000 and 16′000 Da, preferably between 9′000 and 13′000 Da, more preferably between 10′000 and 11′500 Da.

8. The cosmetic composition according to claim 1, wherein the pigment is an inorganic pigment, particularly an inorganic pigment with a colour index between CI 77000 and CI 77999.

9. The cosmetic composition according to claim 1, wherein the pigment is selected from the group consisting of CI 15850, CI 17200, CI 45370, CI 73360, CI 75470, CI 77007, CI 77013, CI 77019, CI 77120, CI 77163, CI 77266, CI 77288, CI 77289, CI 77480, CI 77491, CI 77492, CI 77499, CI 77510, CI 77713, CI 77742, CI 77820, CI 77891, CI 77947 and char coal powder, particularly selected from the group consisting of CI 15850, CI 77491, CI 77492, CI 77499, CI 77510 and CI 77891 and char coal powder.

10. The cosmetic composition according to claim 1, wherein the amount of pigment is in the range of between 0.001 and 15% by weight, particularly between 0.01 and 12% by weight, preferably between 0.05 and 10% by weight, based on the weight of the cosmetic composition.

11. The cosmetic composition according to claim 1, wherein the amount of the mixture of branched and linear saturated C15-C19 alkanes is in the range of between 0.5 and 12.0% by weight, particularly between 1.0 and 10.0% by weight, more preferably between 1.5 and 8.0% by weight, preferably between 2.0 and 6.0% by weight, based on the weight of the cosmetic composition.

12. The cosmetic composition according to claim 1, wherein the cosmetic composition is a decorative cosmetic composition, particularly a lip gloss, a lip ink, a lip stick, a makeup, a foundation, a mascara, an eye liner, an eye shadow, a liquid blush, an eyebrow liner, a concealer, a highlighter or contouring shade.

13. Use of a mixture of branched and linear saturated C15-C19 alkanes, in combination with an ester of a fatty acid and dextrin for increasing the water resistance of a cosmetic composition comprising a pigment.

14. The use according to claim 13, wherein the cosmetic composition comprising a pigment is a decorative cosmetic composition, particularly a lip gloss, a lip ink, a lip stick, a makeup, a foundation, a mascara, an eye liner, an eye shadow, a liquid blush, an eyebrow liner, a concealer, a highlighter or contouring shade.
